(19) United States
(12) Patent Application Publication
(10) Pub. No.: US 2025/0157026 A1
(43) Pub. Date: May 15, 2025

(54) Scaphoid Morphological Assessment by Best Fit Logarithmic Spirals

(71) Applicants: Randolph James Ferlic, Granger, IN (US); Kimberly Kate Ferlic, Granger, IN (US); Richard Anthony de los Santos, Goshen, IN (US); Krissie Marie De los Santos, Goshen, IN (US)

(72) Inventors: Randolph James Ferlic, Granger, IN (US); Kimberly Kate Ferlic, Granger, IN (US); Richard Anthony de los Santos, Goshen, IN (US); Krissie Marie De los Santos, Goshen, IN (US)

(21) Appl. No.: 18/507,034

(22) Filed: Nov. 11, 2023

(51) Int. Cl. G06T 7/00; G06T 7/60

(52) U.S. Cl. CPC G06T 7/0012; G06T 7/60; G06T 2207/10124; G06T 2207/20044; G06T 2207/30008

(57) ABSTRACT

This invention depicts the application of the logarithmic spirals to the morphological assessment of the human scaphoid bone. The intent is to quickly assess if a scaphoid has a displaced fracture or nonunion deformity. This intent is based on the concept that the outer contours of the scaphoid when viewed in the sagittal plane can be modeled by a special case logarithmic spiral called the golden spiral. If this modeling fails it is reasonable to assume that the scaphoid has experienced a displaced fracture or has an established nonunion deformity. The utilization of the mathematical representation of the golden spiral as a diagnostic tool is a novel application allowing for screening of altered scaphoid morphology.

References

US20180342060A1, Medical scan image analysis system, Li Yao, Devon Bernard, Kevin Lyman, Diogo Almeida, Jeremy Howard

### BACKGROUND OF THE INVENTION

### Scaphoid

The scaphoid depicted in FIG. 8 is one of eight small bones that make up the “carpal bones” of the wrist. It is the most frequently fractured of the carpal bones. Its oblique orientation and unique architectural morphology create challenges when trying to radiographically assess. Modern day diagnosis of an acute displaced scaphoid fracture or malaligned established scaphoid nonunion relies upon human visual inspection of radiographic images. A reference tool would be beneficial in order to automate the diagnostic process or enhance human diagnostic capabilities.

### Golden Spiral

The golden spiral is a logarithmic spiral (Eq. 1) whose growth factor is the golden ratio. If a logarithmic spiral is golden, then the growth factor has a value of 0.3063489.

Below is a concise explanation of logarithmic spirals and the derivation of the special case of the golden spiral.

The base equation for a logarithmic spiral is:

$$r ( θ ) = {ae}^{b θ} \qquad Eq . ( 1 )$$

Where r is the resultant radius, a is the initial radius, b is the growth rate, and 0 is the angle.

Solving for b gives:

$$b = \frac{ln ( r / a )}{θ} \qquad Eq ( 1 a )$$

### Assume

$${θ}_{right} = \frac{π}{2} and \frac{r}{a} = ϕ = {e}^{b {θ}_{right}} ,$$

Where ø is the golden ratio. Here we are requiring that

$$\frac{r}{a} = ϕ$$

for a span of

$$\frac{π}{2}$$

radians.

Thus,

$$b = \frac{ln ϕ}{{θ}_{right}} = \frac{ln ϕ}{π / 2} \approx 0.306 \qquad Eq . ( 1 b )$$

Logarithmic spirals that have a growth factor, b=0.3063489, are golden spirals.

FIG. 2 and FIG. 3 show examples of logarithmic spirals. FIG. 2 represents the golden spiral and FIG. 3 represents degenerate variation. Each can be presented using the same expression, but with different b values.

### Application of the Golden Spiral to the Scaphoid

The scaphoid is examined along the mid-sagittal plane (FIG. 9) to look for altered scaphoid morphology as a result of displaced fracture or nonunion deformity. From this perspective, the healthy scaphoid has a morphological contour as seen in FIG. 4.

The golden spiral, mathematically depicted above, provides an accurate representation of a healthy scaphoid in the mid-sagittal plane as seen in FIG. 5. The application of a best fit logarithmic spiral to the contour of an altered scaphoid morphology as a result of a displaced fracture or nonunion deformity (FIG. 6) does not conform to the golden spiral. In this case the growth factor, b, is less than 0.3 (FIG. 7).

### BRIEF DESCRIPTION OF THE DRAWINGS

FIG. 1-Flow Chart

FIG. 2-Logarithmic (Golden) Spiral with b value of 0.3063489

FIG. 3-Logarithmic Spiral with b value of 0.1

FIG. 4-Generic Non-Altered Scaphoid Morphology

FIG. 5-Scaphoid Morphology with Golden Spiral Best Fit with a Growth Factor of 0.3

FIG. 6-Generic Sagittal Plane View of an Altered scaphoid Morphology Due to a Displaced Fracture or Nonunion Deformity

FIG. 7-Generic Sagittal Plane View of an Altered Scaphoid Morphology Due to a Displaced Fracture or Nonunion Deformity with a Best Fit Logarithmic Spiral ˜ b=0.1

FIG. 8-Lateral Plain Film Radiographic Image of Wrist Highlighting the Scaphoid's Irregular Architecture

FIG. 9-Computerized Tomography Image of the Mid-Sagittal Coplanar Section of the Scaphoid

### SUMMARY OF THE INVENTION

The scaphoid must first be radiographically imaged in order to isolate the mid-sagittal plane as seen in FIG. 9. A best fit logarithmic spiral is applied once the image is captured and the outer contour is determined. The conformity or non-conformity of the scaphoid morphology can be assessed based on the growth factor, b, of the logarithmic spiral. As per the description in the Invention Background above, the logarithmic spiral growth factor, b, can be determined. The scaphoid is not morphologically deformed if this exponent value is approximately 0.306, which is nearly the value for the golden spiral described above. If the value is less than 0.3, then it is reasonable to conclude that the scaphoid has altered morphology, potentially reflective of a displaced fracture or nonunion deformity. This result can then be sent to a manual or automatic decision making module to trigger remediation-related events.

### DETAILED DESCRIPTION OF THE INVENTION

This invention does not provide a means to extract the scaphoid contour data. It is expected the data is acquired using whatever means is available to the medical diagnostic system. This invention is intended to offer a software module or function that can be added to an existing medical device data processing system that provides an efficient means to quickly assess a scaphoid (FIG. 8) for displaced fracture or nonunion deformity. The medical data processing system or a clinician can then make a decision based on this assessment. The invention serves as a mathematical tool which can be applied for the radiographic assessment of scaphoid morphology in order to objectively determine the presence of altered morphology.

As described in the summary, the scaphoid (FIG. 8) is the most frequently fractured of the carpal bones. The previous means of evaluation has been human visual review of radiological scans. The purpose of this invention is to provide an automated process to quickly detect abnormalities in the scaphoid morphology. It relies on the principle of applying a best fit logarithmic spiral to a given data set. The mathematical representation of the logarithmic spiral is presented in Eq. 1. This invention relies on contour data of a scanned scaphoid when viewed along the mid-sagittal plane (FIG. 9). A best fit logarithmic spiral is then applied.

Generically, when a logarithmic spiral is applied to a data set two constants can be varied to find the spiral with the lowest error: the initial radius of the spiral, as depicted as a and the growth factor, b, in Eq 1. For the purposes of this invention a is assumed to be 1 and the data set is scaled accordingly. Only the growth factor is adjusted to find the best fit logarithmic spiral that represents the scaphoid contour position data.

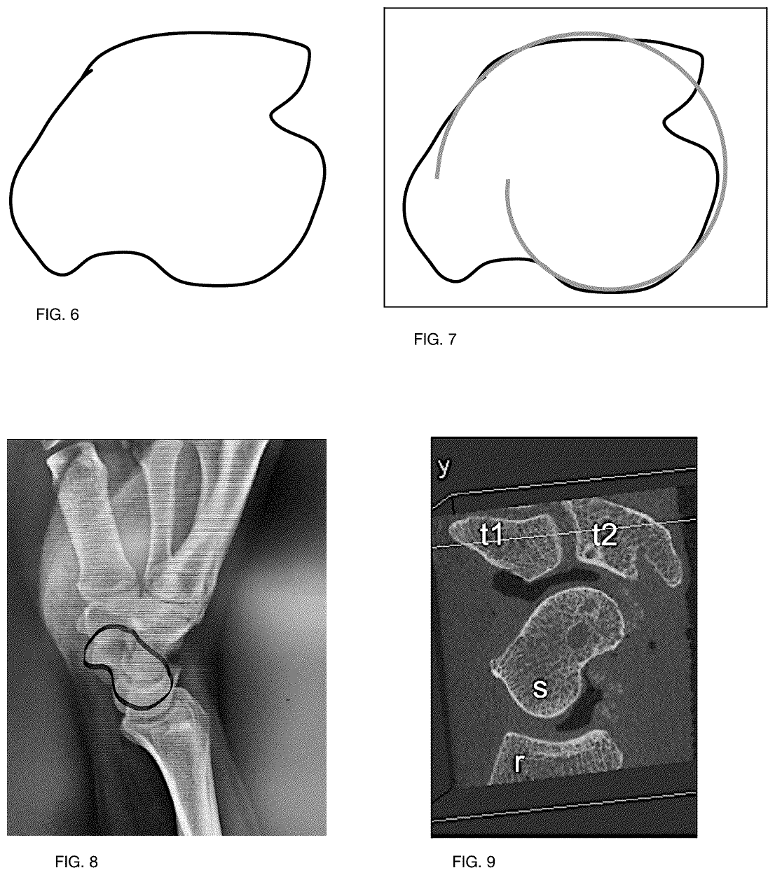

A decision can be made on the conformation of the scaphoid based on the value determined for the growth factor. A morphologically normal scaphoid (FIG. 4) has a contour that can be modeled by a golden spiral, with a growth value where b=0.3063489 (FIG. 5). A scaphoid with a displaced fracture or nonunion deformity (FIG. 6) will have a much lower growth value, where b is between 0.1 and 0.2 (FIG. 7).

Although other solutions exist for detecting fractures, this invention is novel. Yao demonstrates that the “metacarpal and scaphoid are listed as suspicious for fracture, with the confidence presented as one out of four blocks shaded.” This is based on the use of learning algorithms that are presented with “a plurality of medical scans” leading to the algorithm detecting “an inferred abnormality.” The invention depicted in our present patent is novel in that it does not rely on training set data or learning algorithms. It uses the application of a best fit logarithmic spiral to the scaphoid contour data and bases the detection of a fracture or a deformity on the growth factor b, as explained above.

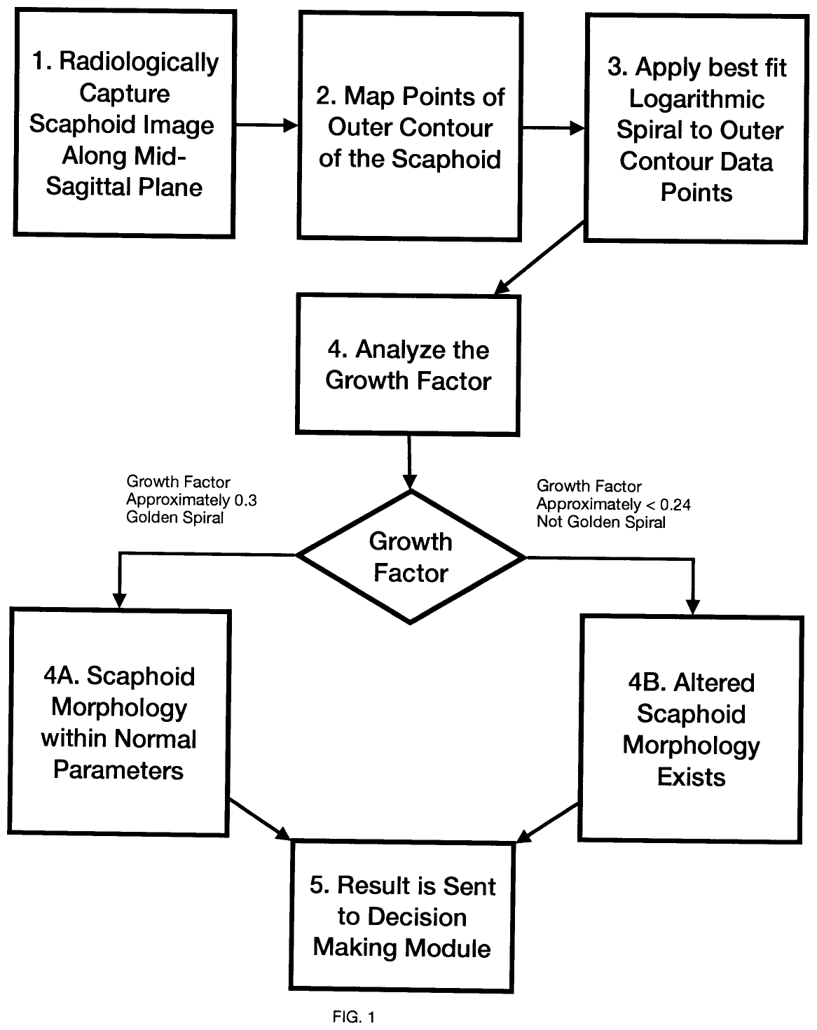

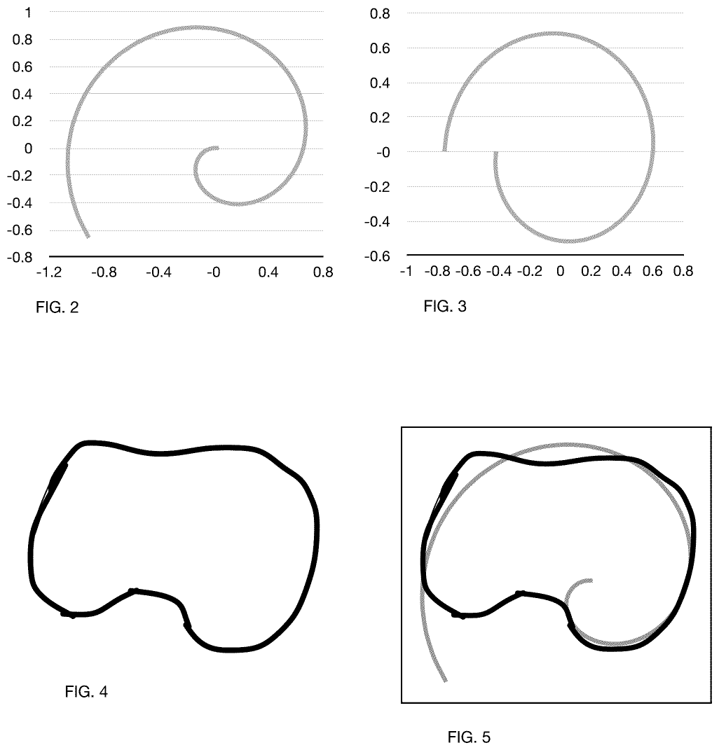

Below are the precise steps of the invention as depicted in the flow chart (FIG. 1).

### Steps for the Application of the Invention

1. The scaphoid is radiographically imaged in order to isolate the mid-sagittal plane.
2. The outer contour data points of the scaphoid are then derived using edge or boundary detection algorithms.
3. A best fit logarithmic spiral is then applied to the points.
4. The growth factor is analyzed:
   A. If the growth factor represents the golden spiral, then scaphoid morphology is within normal parameters.
   B. If the growth factor does not represent the golden spiral, then altered scaphoid morphology exists.
5. The result is sent to a decision making module. For example, this module can be a data processing software or an onscreen view for a doctor or clinician to review.

1. The scaphoid can be characterized geometrically.

1. The golden spiral provides an accurate representation of the mid-sagittal coplanar section of the scaphoid.

2. The golden spiral can be used to model the standard morphological contours of a scaphoid when viewed along the mid-sagittal plane.

3. The golden spiral can be utilized as a diagnostic tool in order to assess scaphoid morphology when viewed in the mid-sagittal plane.

4. This invention assesses if a scaphoid has suffered a displaced fracture or has a nonunion deformity when viewed along the mid-sagittal plane.

5. Altered scaphoid morphology secondary to a displaced fracture or an established nonunion results in a smaller logarithmic spiral exponent reflective of a rounder profile disallowing golden spiral representation.

6. If the morphological contours, when viewed along the sagittal plane, do not conform to the golden spiral then the scaphoid may have a displaced fracture or nonunion deformity.
